(19) United States
(12) Patent Application Publication
(10) Pub. No.: US 2026/0357218 A1
(43) Pub. Date: Nov. 12, 2026

(54) A NOVEL EXPRESSION PLATFORM FOR STABLE AND HIGH TITER MASS PRODUCTION OF RECOMBINANT PROTEIN

(71) Applicant: ILKO ILAC SANAYI VE TICARET A.S., Istanbul (TR)

(72) Inventors: Hatice ONCEL, Istanbul (TR); Busra SIMSEK, Istanbul (TR); Duygu DAGLIKOCA, Istanbul (TR); Dilan KARABULUT, Istanbul (TR); Beyzanur DENIZ, Istanbul (TR); Aylin OZKAN, Istanbul (TR); Onur PINARBASLI, Ankara (TR); Nagehan SARRACOGLU, Ankara (TR)

(73) Assignee: ILKO ILAC SANAYI VE TICARET A.S., Istanbul (TR)

(21) Appl. No.: 19/124,349

(22) PCT Filed: Oct. 31, 2022
(86) PCT No.: PCT/IB2022/060504

§ 371 Date: Apr. 25, 2025

(51) Int. Cl. C12N 5/071; C07K 16/00; C12N 9/06; C12N 15/85

(52) U.S. Cl. CPC C12N 5/0682; C07K 16/00; C12N 9/003; C12N 15/85; C07K 2317/14; C12N 2510/00; C12N 2800/107; C12N 2830/46; C12N 2830/50; C12N 2840/203; C12Y 105/01003

(57) ABSTRACT

The object of the present invention is to provide an expression platform, including a vector containing DHFR, at least one promoter, and a DNA element, such as UCOE, IRES or a combination thereof. Another object of the present invention is to provide a method utilizing the expression platform to produces cells with unique genetic background capable of robust and high level expression of a target protein, and a method for high yield recovery of the produced target protein.

### CROSS REFERENCE TO RELATED APPLICATIONS

This application is a National Stage of International Application No. PCT/IB2022/060504 filed Oct. 31, 2022.

### INCORPORATION BY REFERENCE OF SEQUENCE LISTING

The instant application contains a Sequence Listing which has been filed electronically in xml format and is hereby incorporated by reference in its entirety. Said xml copy, created on Apr. 24, 2025, is named Q304146_Sequence_listing_as_filed.xml and is 11.5 bytes in size.

### TECHNICAL FIELD

The present invention relates to an expression platform including an expression vector which provides for high gene copy number and enhanced production of recombinant proteins or peptides.

In particular, the present invention relates to an expression platform, including a vector containing nucleotide sequence encoding a target protein or peptide, such as, for example, an antibody; a nucleotide sequence encoding Dihydrofolate reductase (DHFR), which provides high enhancement of gene copy number; a promoter selected from the CMV promoter, EF1α promoter, SV40 promoter or a combination thereof, and a DNA element, such as a ubiquitous chromatin opening element (UCOE), which increases random integration efficiency and provides genetic stability and high titer mass production of the recombinant protein via effectively keeping the corresponding promoter active, and a nucleotide sequence encoding an internal ribosome entry site (IRES), which allows for the cap-independent translation initiation. As compared with some existing cell expression vectors, the vector platform of the present invention enables a method for effective production of single cells and clonal populations of cells with unique genetic background ensuring robust production of recombinant proteins or peptides and high recovery upstream and downstream of the production process.

### BACKGROUND ART

The recombinant production of proteins and peptides, including antibodies, in eukaryotic cells involves the creation of expression systems. Expression systems for the production of as recombinant proteins, such as biological therapeutics or biopharmaceuticals, generally consist of a nucleic acid vector construct encoding the desired recombinant protein, and a chosen host cell. The vector is introduced into the host cell and the endogenous cell machinery is utilized for the production of the desired recombinant protein, e.g., a biological therapeutic. The intricacies in establishing an efficient and reliable expression system for the production of approvable biological therapeutics are manifold.

A number of approaches exist for the design and construction of expression vectors, and the process typically requires substantial trial and error experimentation before reasonable levels of protein are produced. A significant consideration in the design process concerns the use of intron sequences in the construction of the vector.

In one approach, an entire gene sequence may be utilized as it occurs naturally, i.e., containing the full complement of both intronic and exonic sequences. In such a case, it is expected that post-transcriptional splicing machinery within the cell will excise intronic sequences to yield a mature mRNA containing only exonic sequences of the gene.

Another approach is to utilize the nucleic acid sequence corresponding to the cDNA of the gene only. In this case, it is predicted that no splicing events occur and the pre-mRNA sequence is substantially the same as the mRNA sequence in protein coding content.

In yet another approach, vector construction involves the selection and placement of introns not normally associated with the original gene sequence.

A vector has an ‘origin of replication’; a stretch of DNA that ensures the vector gets replicated (copied) by the host bacterium. Often, it also contains a promoter sequence so that the introduced gene can be expressed (and a protein produced).

Plasmids are extrachromosomal self-replicating cytoplasmic (usually circular) DNA elements found in prokaryotes and, less commonly, in eukaryotes, which may be used as vectors. They come in various forms, such as simple plasmids used for direct transformation and fosmids for phage transduction. There are other forms of circular DNA, each with different desirable properties (e.g., larger insertion size, low copy number, phage-compatibility, etc.). They are sometimes called ‘high capacity vectors’ because their insertion sizes are greater than those of simple plasmids.

Bacterial artificial chromosomes (BACs) contain some regions derived from a special plasmid called the F (fertility) factor: the region containing an origin of replication as well as genes that ensure its precise segregation during bacterial cell division. A great advantage of BAC vectors is the large insertion size (100-200 kb). But the very large insertion can be a problem, in that it cannot be manipulated by restriction enzymes.

Yeast artificial chromosome (YAC) has a cloning capacity up to 3000 kbp. It is introduced into the yeast cells by electroporation, and then it is maintained as a linear DNA like a chromosome. It is replicated along with other chromosomes in yeast and its copy number of one is maintained after cell division.

Some viruses (e.g., Adenovirus, Lentivirus and Baculovirus) and bacteria (e.g., Agrobacterium tumefaciens) are reliable vectors for stable transfection of eukaryotic cells. Viral vectors are generally genetically engineered viruses carrying modified viral DNA or RNA that has been rendered noninfectious, but still contain viral promoters and also a transgene, thus allowing for transcription of the transgene under the control of a viral promoter. However, because viral vectors frequently are lacking infectious sequences, they require helper viruses or packaging cell lines for large-scale transfection. Viral vectors are often designed for permanent incorporation of the insert into the host genome, and thus leave distinct genetic markers in the host genome after incorporating the transgene. For example, retroviruses leave a characteristic retroviral integration pattern after insertion that is detectable and indicates that the viral vector has incorporated into the host genome.

The choice of an expression system for the production of recombinant proteins depends on many factors, including cell growth characteristics, desired expression levels, intracellular or extracellular expression, post-translational modifications and biological activity of the protein of interest, as well as regulatory issues and economic considerations in the production of therapeutic proteins. Key advantages of mammalian cells over other expression systems, such as bacteria or yeast, are their ability to carry out proper protein folding, complex N-linked glycosylation and authentic O-linked glycosylation, as well as a broad spectrum of other post-translational modifications. Due to these advantages, eukaryotic and, in particular, mammalian cells are currently the expression platform of choice for producing complex therapeutic proteins.

Cell culture studies are widely used in pharmaceutical, medical and biotechnological research. For the production of recombinant proteins, cell culture conditions should be standardized to ensure optimum performance and stability of cell cultures. It is necessary to constantly control different parameters and conditions during cell culture to ensure that cells grow properly and optimally produce the desired recombinant protein.

As mentioned above, mammalian cells have become the dominant system for producing recombinant protein products for clinical application because of their capacity to properly fold and assemble proteins and add humanlike posttranslational modifications. In fact, all cell lines used for biopharmaceutical protein production so far have originated from mammals (Non-Patent Literature 1). However, mammalian cell line development is often very time-consuming. In addition, the mammalian cell culture process is hampered by low yields and unstable expression by the cells (Non-Patent Literature 2). Productivity and stability of expression are the prerequisites for developing commercially viable processes. Therefore, the ultimate goal of cell line development is to obtain clonal cell lines that secrete the protein of interest with high specific productivity (Qp), and at consistently high levels over an extended number of cell generations, allowing for scale-up and cost-efficient manufacturing. Expression vector and cell line engineering are the keys for achieving this goal.

The difficulty of protein synthesis is the high cost of production due to low production yields. The production yield of a clone depends upon selection of several factors: external factors, such as culture conditions (media components, temperature, pH, etc.) and downstream purification process; and internal factors, such as selection of vector and its regulatory elements like promoter, transcription or translation enhancing elements, and other elements, and their appropriate orientation, and the choice of a suitable host cell. Mammalian cell is the most promising expression system to obtain high expression of recombinant therapeutic proteins as it has a natural capacity of glycosylation. Also, post-translational modifications in such expression systems are more likely to resemble those found in proteins expressed in human cells, thus ensuring the appropriate physiological activity of the recombinant protein. However, the expression levels in eukaryotic cells are also highly dependent on another internal factor, i.e., the integration site of the recombinant expression construct comprising the gene of interest in the genome of the host cell.

As discussed above, mammalian cell culture is a preferred technique in the industry for overexpressing the target protein. Because proteins with industrial value are mostly human or animal derived proteins, and specific protein modification mechanisms (glycosylation, phosphorylation, amidation) are carried out easily in animal cells. The high cost of protein production and the large amount of time and high expense required for cell line creation are known limitations of this technique (Non-Patent Literature 3). The animal cells currently used in the industry are Chinese Hamster Ovary (CHO) cells, Baby Hamster Kidney (BHK) cells and myeloma cells, where the target protein is expressed by transfecting an expression vector into the cells.

CHO cells are an epithelial cell line often used in biotechnological research and commercially in the production of recombinant therapeutic proteins. It is used in genetics, toxicity screening, nutrition and gene expression studies, and to express recombinant proteins. CHO cells are the most widely used mammalian hosts for the industrial production of recombinant protein therapeutics (Non-Patent Literature 4).

CHO cells can produce proteins with complex glycosylation and other post-translational modifications (PTMs) similar to those produced in humans. They can be easily grown in large-scale cultures and have high viability, making them ideal for GMP protein production. In addition, CHO cells are tolerant of changes in parameters such as oxygen levels, pH, temperature or cell density (Non-Patent Literature 5). Most of the genetic manipulation done in CHO cells is done in cells lacking the DHFR enzyme.

Gene amplification is a routinely used strategy in animal cell expression systems. There are two commonly used amplification systems. These are the DHFR based amplification and glutamine synthetase based amplification. Both significantly increase the recombinant protein yield of animal cell lines. Despite the advantage of improving protein production, gene amplification systems have the disadvantage in that they require multiple rounds of gene amplification and the use of high concentrations of methotrexate (MTX), which is time consuming. Prolonged subculture of cell lines leads to gene loss and unstable expression.

Despite certain challenges, selection schemes based on DHFR deficient cells remains one of the standard methods for generating transfected CHO cell lines for the production of recombinant therapeutic proteins.

Non-Patent Literature 6 and Patent Literature 1 provide reports of high level of a foreign gene being co-expressed in the animal cell when it is inserted in the vicinity of the DHFR gene in the expression vector.

Gene amplification in CHO cells begins with molecular cloning of the gene of interest and the DHFR gene into a single mammalian expression system. Plasmid DNA carrying the two genes is then transfected into cells and the cells are grown under selective conditions in a thymidine-free medium. The growth rate and level of recombinant protein production of each cell line vary greatly. Evaluation of several hundred candidate cell lines may be necessary to obtain several stable transfected cell lines with the desired phenotypic characteristics (Non-Patent Literature 7 & Non-Patent Literature 8).

Currently used methods for creating mammalian cell lines for expression of recombinant proteins suffer from several drawbacks (Non-Patent Literature 9). Episomal systems allow for high expression levels of the recombinant protein, but frequently are only stable for a short time period (Non-Patent Literature 10). Mammalian cell lines containing integrated exogenous genes are somewhat more stable, but there is increasing evidence that stability depends on the presence of only a few copies or even a single copy of the exogenous gene.

Efficiency of the system itself depends on a large variety of factors including the design of the vector and the choice of the host cell. The strategic combination of regulatory elements, selection markers and stability elements within the vector sequence has to balance simplicity of manipulation and application of the vector with the need for high yield production of the desired biological therapeutic. It is a particular challenge to develop methods for making host cells that stably express recombinant proteins over extended periods of time and at high levels.

Despite the progress, protein expression levels in mammalian cells are relatively low and often unstable during the time of development, resulting in high development and production costs for therapeutic proteins.

Current biomanufacturing processes require cell lines to be capable of achieving unclumped and robust growth in suspension culture, stably and productively integrating heterologous DNA, producing high recombinant protein concentrations in a given system, and performing desired posttranslational modifications with uniform product characteristics (Non-Patent Literature 10, Non-Patent Literature 11).

In addition, the availability of a suitable expression system and the speed at which a high-yielding clone can be obtained may also influence the choice of the cell line. For regulatory approval, production cell lines must be well characterized and genetically stable; thus, using a cell type familiar to regulators will limit the intensity of their scrutiny. Some cell line specific differences can significantly affect the performance of a production system; for example, glycosylation of a given protein varies with the type of mammalian cells used, and even two subclones from the same parental line can differ greatly in metabolic requirements.

Although the properties of some of the regulatory elements and internal factors used in the expression vector are well understood, the ability of their combinations to reliably and efficiently achieve high expression of the target proteins is not absolutely predictable. Some combinations give very poor expression in comparison with others. For example, as reported in Non-Patent Literature 12, using an expression vector consisting of a combination of SR a promoter, AMY RNA leader sequence and DHFR achieved an erythropoietin (EPO) expression of only 45 IU/ml (equivalent to 0.346 μg/ml). Patent literature 2 reports levels of EPO of 750 to 1470 U/million cells/48 Hrs (or 375 to 735 U/million cells/24 Hrs) using an expression vector consisting of another combination of elements namely, SV40 and poly-adenylation (polyA) sequence, and DHFR. Still another expression vector reported in Patent Literature 3 and consisting of a combination of EF-1 promoter and apoB SAR elements, reportedly achieved an expression of 1500 to 1700 IU of EPO/million cells/24 Hrs. For other recombinant proteins, such as TNFR-IgGFc (Enbrel), an expression vector containing a combination of CMY promoter, TPL, YA I & II, and DHFR has been reported (Patent Literature 4).

### SUMMARY OF THE INVENTION

### Technical Problem

Surprisingly, despite the tremendous amount of knowledge generated in this area over the last two decades, even today a person skilled in the art cannot simply choose a combination of internal factors and regulatory elements to design an expression vector that would give guaranteed high expression of the target protein or peptide. A particular element when added to a combination of other elements may not provide any significant additive or synergistic effect to the expression potential of the vector. Therefore, the process of developing a novel expression vector that would give high level of protein expression is challenging and still requires empirically testing many possibilities. Accordingly, there exists a need for cell culture process for improving recombinant protein production, particularly, for the large-scale production of therapeutic proteins stable over extended periods of time.

### Solution to Problem

There are three especially important parameters that need to be considered during cell line development for the production of biological products. These are stability, productivity and quality. These three parameters can be controlled through the mammalian expression vector that enables the target gene to be transferred into the host cell. For this reason, this invention aims to design and create a mammalian expression vector carrying elements that can provide good results according to these parameters.

The present inventors developed an expression platform, which allows obtaining large amount of a target protein using a recombinant vector containing the human-derived DHFR gene operably linked to the mouse-derived DHFR promoter. This system can effectively amplify the target gene at a lower concentration of MTX.

The vector of the present invention enables efficient selection of a cell line clone containing the DHFR gene and the target gene amplified under very low concentrations of MTX compared to the existing animal cell expression vectors. In fact, it has been determined that there is no need to use MTX in some scale-up processes according to the present invention. In the examples provided herein, MTX was only used in limiting dilution cloning (LDC) step. Accordingly, cell culture process may be performed in the presence of low methotrexate (MTX) or in the absence of MTX. Cell culture process indicates cell line production, followed by process and media optimization in small-scale systems, including shaker flasks and bench-scale bioreactors and scale-up process.

Some vectors of the present invention contain the IRES element, which allows for two proteins to be expressed from a single mRNA, which may be important for protein yield. The CMV promoter is susceptible to gene silencing in CHO cells due to epigenetic events such as DNA methylation and histone modifications.

In one aspect, the present disclosure provides a vector design system for improving the target protein or peptide expression and/or production. In some aspects, the protein or peptide is a recombinant protein or peptide. In some aspects, the target protein is an antibody.

In another aspect, the present disclosure provides a recombinant nucleic acid encoding the target protein or peptide and the genetic elements necessary for the expression of the target protein or peptide in a host cell.

In another aspect, the present disclosure provides an expression vector for the production of the protein or peptide including an antibody.

In another aspect, the present invention provides an expression vector for generating a stable cell expressing the recombinant protein or peptide.

In another aspect, the present disclosures provides a host cell, for example, a eukaryotic host cell, containing one or more of the foregoing nucleic acid molecules and/or vectors, e.g., expression vectors. The host cell can be transiently or stably transfected with the nucleic acid sequences of the invention. The cell may be a mammalian cell, such as, for example, a CHO cell.

In another aspect, the present disclosure provides a system for culturing such cells according to the present disclosure to produce the recombinant proteins or peptides on a large-scale.

In another aspect, the present disclosure provides a selection system for selecting host cells expressing the target protein or peptide, which allows obtaining high yield of the target protein or peptide.

In another aspect, the present disclosure provides an expression system for the production of recombinant products, such as proteins, peptides, antibodies, which includes a host cell transected with a vector according to the present disclosure.

The vector of the present disclosure may be an expression vector and may include one or more of the foregoing modified nucleic acid elements. The vector can additionally include a nucleotide sequence that enhances one or more of: replication, selection, mRNA transcription, mRNA stability, protein expression or protein secretion, in a host cell. For example, the vector may include nucleotide sequences responsible for replication or enhancer expression, enhancer promoter elements, nucleotide sequences encoding a leader sequence, a gene encoding a selectable marker (e.g., DHFR), an internal ribosomal entry site sequence (IRES), and polyadenylation sequences.

In some aspects, the vectors (e.g., expression vectors) are modified to reduce or eliminate mis-spliced and/or intron read-through by-products and/or to enhance recombinant protein expression.

In another aspect, the present disclosure provides a vector design comprising a nucleic acid sequence encoding an antibody (e.g., a recombinant antibody), or a fragment thereof, having reduced (e.g., substantially free of) mis-spliced and/or intron read-through products for the large-scale production of therapeutic proteins stable over extended periods of time.

In another aspect, the present disclosure utilizes a novel combination of elements to develop a novel vector platform which provides for a synergistic effect of these elements resulting in high expression of desired proteins.

In some aspects, the expression vector of the present disclosure contains the following operably linked elements:
a nucleotide sequence encoding one or more target proteins or peptides;
one or more terminator sequence;
one or more promoter;
a chromatin opening element (UCOE), a nucleotide sequence encoding an internal ribosome entry site (IRES) or a combination thereof, and
a nucleotide sequence encoding a dihydrofolate reductase (DHFR) as a selection marker.

In some aspects, the expression vector is a bicistronic vector or a dual promoter vector, where the promoters may be the same or different from each other.

In some aspects, the target protein or peptide is a monoclonal antibody. In some aspects, the target protein or peptide is an antibody light chain (LC) and an antibody heavy chain (HC). In some aspects, the expression of the LC is controlled by the first promoter and the expression of the HC is controlled by the second promoter.

In some aspects, the DHFR is a human DHFR, and the nucleotide sequence encoding the human DHFR is operably linked to a mouse-derived DHFR promoter.

In some aspects, the first promoter and the second promoter are each, independently, a CMV promoter or a EF1-α promoter.

In some aspects, the UCOE comprises the sequence of SEQ ID NO: 4.

In some aspects, the nucleotide sequence encoding the IRES comprises the sequence of SEQ ID NO: 5.

In some aspects, the DHFR comprises the sequence of SEQ ID NO: 6.

In some aspects, the expression vector further comprises a first multiple cloning site (MCSI) and a second multiple cloning site (MCSII).

In some aspects, the one or more terminator sequence is a polyA signal sequence and a polyA SV40 terminator sequence.

In some aspects, the elements in the expression vector are arranged in the following order in 5′ to 3′ direction:
the UCOE, EF1-α promoter, MCSI, the nucleotide sequence encoding the IRES, MCSII, the polyA signal sequence, SV40 terminator sequence, the nucleotide sequence encoding the DHFR, and the polyA signal sequence.

In some aspects, the elements in the expression vector are arranged in the following order in 5′ to 3′ direction:
the UCOE, CMV promoter, MCSI, the nucleotide sequence encoding the IRES, MCSII, the polyA signal sequence, the SV40 terminator sequence, the nucleotide sequence encoding the DHFR, and the polyA signal sequence.

In some aspects, the SV40 terminator sequence comprises a sequence of SEQ ID NO: 1.

In some aspects, the present disclosure provides a cell or a population of cells transfected with the expression vector of the present disclosure. In some aspects, the cells are mammalian cells.

In some aspects, the cells are Chinese Hamster Ovary (CHO) cells.

In some aspects, the present disclosure provides a mammalian cell culture process to propagate the cells of the resent disclosure performed in the presence of methotrexate (MTX) or in the absence of MTX.

In some aspects, the present disclosure provides a method of producing a target protein or peptide by culturing the cells of the present disclosure under conditions for expressing the target protein or peptide in a culture medium.

In some aspects, the cells of the present disclosure are clones stably expressing the target protein or peptide.

In some aspects, the target protein or peptide is a monoclonal antibody, antibody heavy chain, or antibody light chain, or a combination thereof.

### DEFINITIONS

In the context of the present invention, the words “comprise”, “comprising” and the like are to be construed in their inclusive, as opposed to their exclusive, sense, that is in the sense of “including, but not limited to.”

As used herein, the term “stable,” when used in reference to genome, refers to the stable maintenance of the information content of the genome from one generation to the next, or, in the particular case of a cell line, from one passage to the next. Accordingly, a genome is considered to be stable if no gross changes occur in the genome (e.g., a gene is deleted or a chromosomal translocation occurred). The term “stable” does not exclude subtle changes that may occur to the genome such as point mutations.

“Operably linked” refers to a juxtaposition of two or more components, wherein the components so described are in a relationship permitting them to function in their intended manner. For example, a promoter and/or enhancer are operably linked to a coding sequence, if it acts in cis to control or modulate transcription of the linked sequence. Generally, but not necessarily, the DNA sequences that are “operably linked” are contiguous and, where necessary to join two protein encoding regions such as a secretory leader and a polypeptide, contiguous and in (reading) frame.

A “selection marker” which is expressed by the introduced polynucleotide allows under appropriate selective culture conditions the selection of host cells expressing said selectable marker. A selection marker is preferably a biomolecule, in particular a polypeptide.

A “vector” according to the present invention is a polynucleotide capable of carrying at least one polynucleotide fragment. The term “expression vector” includes a specific type of vector wherein the nucleic acid construct is optimized for the high-level expression of a desired protein product. Expression vectors often have transcriptional regulatory agents, such as promoter and enhancer elements, optimized for high-levels of transcription in specific cell types and/or optimized such that expression is constitutive based upon the use of a specific inducing agent. Expression vectors further have sequences that provide for proper and/or enhanced translation of the protein. As known to those skilled in the art, such vectors may easily be selected from the group consisting of plasmids, phages, viruses, and retroviruses. The term “expression cassette” is a distinct component of vector DNA consisting of a gene and regulatory sequence to be expressed by a transfected cell. In each successful transformation, the expression cassette directs the cell's machinery to make RNA and protein(s). A vector functions like a molecular carrier, delivering fragments of nucleic acids respectively polynucleotides into a host cell. It may comprise at least one expression cassette comprising regulatory sequences for properly expressing a polynucleotide incorporated therein. Polynucleotides (e.g. encoding the product of interest or selectable markers) to be introduced into the cell may be inserted into the expression cassette(s) of the vector in order to be expressed therefrom.

The term “DHFR (Dihydrofolate reductase)” used herein refers to an enzyme that reduces dihydrofolic acid to tetrahydrofolic acid, which is a key enzyme for nucleic acid synthesis and an essential enzyme for cell growth.

The term “intron” as used herein includes a segment of DNA that is transcribed, but removed from the RNA transcript by splicing together the sequences (exons) on either side of it. Introns are considered to be intervening sequences within the protein coding region of a gene and generally do not contain information represented in the protein produced from the gene.

The term “antibody” refers to a protein having a four-polypeptide chain structure consisting of two heavy and two light chains, said chains being stabilized, for example, by interchain disulfide bonds, wherein the immunoglobulin or antibody has the ability to selectively or specifically bind an antigen.

The term ‘monoclonal antibody’ refers to an antibody obtained from a population of substantially homogeneous antibodies, i.e., the individual antibodies comprising the population are identical and/or bind the same epitope.

The term “commercial product” can also refer to the commercially available CHO cell line (e.g., CHO cell line produced by CATALENT).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term ‘host cell’ refers to cells into which exogenous nucleic acid has been introduced, including the progeny of such cells.

The term ‘SV40 terminator sequence’ refers to SV40 polyA region. SV40 polyA is a region of the SV40 (Simian virus 40) genome where transcripts coming from both directions terminate. Hence, it functions as a transcription terminator and poly A signal in either orientation.

### Advantageous Effects of the Invention

The present invention describes an expression vector for recombinant protein for the generation of a stable cell. The unique alterations are particularly suitable for recombinant protein production, particularly monoclonal antibody, but may be used for any protein of interest. Introns and exons are nucleotide sequences within a gene. Introns are removed by RNA splicing as RNA matures, meaning that they are not expressed in the final messenger RNA (mRNA) product, while exons go on to be covalently bonded to one another in order to create mature mRNA. The nucleic acid molecule reduces or eliminates the intron reading (IRT) by-products of a desired protein or peptide relative to a naturally occurring sequence. Moreover, the expression vectors of the present invention having introns and exons with altered natural operative associations demonstrate not only reduced or eliminated IRT by-products but also increased stability and protein expression levels relative to vectors designed using standard art recognized techniques.

The features and advantages of the present invention may be summarized as follows:
(1) The vector of the present invention is unique in the combination of regulatory elements it contains. In this way, it ensures optimum production of recombinant protein.
(2) The vector of the present invention provides effective selection of a cell line clone with the amplified DHFR gene and foreign gene under very low concentrations of MTX compared to the currently available animal cell expression vectors.
(3) The present invention has advantageous effects of allowing to use lower MTX concentration and reducing cost due to increased cell growth rate and productivity.
(4) The present invention provides good results in terms of biosimilarity, unlike existing commercial product. Biosimilarity may not always be achieved with existing products.
(5) The risk of the present invention in terms of stability is very low.
(6) Existing commercial systems may have to be produced with media and supplements specific to these systems. This situation affects the cost and makes it difficult to create the process suitable for the working budget. However, in the present invention, this drawback has been eliminated and various media and supplements can be used.

In the present invention, even if random integration occurs, integration efficiency and stability are increased with UCOE.

### BRIEF DESCRIPTION OF DRAWINGS

FIG. 1 Schematic representation of a dual promoter vector.

FIG. 2 Schematic representation of a bicistronic vector.

FIG. 3 The secondary structure of the IRES complex.

FIG. 4 CMV promoter sequence (SEQ ID NO: 3).

FIG. 5 A2UCOE structure.

FIG. 6 A2UCOE sequence (SEQ ID NO: 4).

FIG. 7 IRES sequence (SEQ ID NO: 5).

FIG. 8 Sequence fragment showing ATG-11 and ATG-12 translation initiation sites in IRES (SEQ ID NO: 7).

FIG. 9 Charge Profile (CEX) graphs comparing change profiles of the originator molecule with the molecule produced by Catalent cell line and the molecule produced by Example-1E.

FIG. 10 Charge profile (cIEF) graphs without CpB and with CpB comparing the change profiles of the originator molecule and the molecule produced by Example-1E.

FIG. 11 EF1α sequence (SEQ ID NO: 2).

FIG. 12 SV40 terminator sequence (SV40) (SEQ ID NO: 1).

FIG. 13 DHFR sequence (SEQ ID NO: 6).

### DESCRIPTION OF EMBODIMENTS

### [Host Cells]

The mammalian host cells of the present invention may be any of those commonly used in the art for expressing recombinant proteins, polypeptides or peptides. For example, host cells may be Chinese Hamster Ovary (CHO) cells such as CHO-K1, CHO-DG44 DHFR− and CHO-S. These include both adherent and suspension cell lines.

Chinese hamster ovary cell lines are commonly used as a host for production of recombinant proteins both in research and in the biotech industry. Recombinant cell lines are generated through random integration of multi-cistronic plasmid vectors containing the genes of interest and selection marker genes into the host genome. The recombinant cell lines require several rounds of limiting clonal dilution to isolate stable high expressing clones. Stable high expression of the genes of interest is a rare and desired trait in recombinant clonal cell lines. Despite being clonal, cell lines may eventually become heterogeneous and lose productivity.

### [Vectors]

Any suitable expression vectors may be used in the present invention. Preferably, dual promoter and bicistronic vectors are used in the present invention.

A schematic representation of a dual promoter vector is depicted in FIG. 1. The expression of two different target genes is controlled by two different promoters. If the dual promoter vector is used to express an antibody, the expression of the heavy chain and the light chain may be controlled by two separate promoters.

A schematic representation of a bicistronic vector is depicted in FIG. 2. The expression of two different target genes is controlled by the same promoter. If a bicistronic vector is used to express an antibody, the expression of the heavy chain and the light chain is controlled by the same promoter region. In order to control the heavy and light chain by a single promoter, the IRES (Internal ribosome entry site) sequence may be used in bicistronic vector models.

### [Vector Designs Incorporating Regulatory Elements]

The following sections describe some of the DNA elements that may be used in expression systems of the present invention. Other suitable DNA elements with the same or similar functions known to one of ordinary skill in the art may also be used in the embodiments of the present invention.

### [Promoters]

Below are some examples of the promoters that may be used in the present invention. Other suitable promoters may also be used as would be understood by one of ordinary skill in the art.

### Simian Virus 40 Promoter

Mammalian expression plasmids are primarily used to create mRNA and the commonly used mammalian terminators (SV40, hGH, BGH, and rbGlob) include the sequence motif AAUAAA which promotes both polyadenylation and termination. Out of those listed, the SV40 late polyA and rbGlob polyA are thought to be more efficient in terminating transcription due to the presence of additional helper sequences. The polyA sequence generally promotes transcript stability or degradation in eukaryotes and prokaryotes respectively. While the SV40 sequence is a terminator sequence that signals the end of a transcriptional unit. Terminator sequences also have a role in RNA processing and stability. The genes of interest are usually placed downstream of strong viral promoters, such as simian virus 40 (SV40) terminator sequence (SEQ ID NO: 1) and cytomegalovirus (CMV) promoter (SEQ ID. NO: 3), to obtain high levels of expression. The CMV promoter is a popular choice as it is a relatively strong promoter compared to the SV40 terminator sequence and because it is common in commercially available plasmids, though other promoters such as human elongation factor one alpha (EF-1α) and Chinese hamster elongation factor one (CHEF-1) promoters are stronger.

### Human EF1 Alpha Promoter

Human EF1 alpha (EF-1α, gene symbol EEF1A1) (SEQ ID NO: 2) is a continuously active promoter in a wide variety of cell types. Some studies have shown that promoters of endogenous mammalian genes such as EEF1A1 may be more resistant to silencing than viral promoters. The EF-1α promoter, used in conjunction with the flanking regions of the CHO EF-1α gene, is more active in CHO cells compared to the CMV and SV40 promoters (Non-Patent Literature 3).

### CMV Promoter

CMV promoter (SEQ ID NO: 3) is the most widely used promoter in the biopharmaceutical industry in customized and commercial vectors due to its continuous and high expression. However, CMV promoter can be silenced over time in some cell types, causing heterogeneity between transfected cells (Non-Patent Literature 4 & Non-Patent Literature 5). To prevent transcriptional silencing due to methylation, the main CpG island element (core CpG island element (IE)) is integrated into the promoter (Non-Patent Literature 7).

### [Ubiquitous Chromatin Opening Elements (UCOEs)]

UCOEs are cis-acting epigenetic regulatory elements derived from the promoter regions of housekeeping genes. They are CpG islands that do not contain methylation and prevent heterochromatin formation and silencing of the transgene by reducing DNA methylation (Non-Patent Literature 8).

The A2UCOE region from the human HNRPA2B1-CBX3 locus is one of the most efficient UCOEs, and its incorporation into expression vectors has been shown to increase transgene expression levels in mammalian cells (Non-Patent Literature 13 & Non-Patent Literature 16). The A2UCOE with a sequence of SEQ ID NO: 4 is one example of the A2UCOE that may be used in the present invention. Other suitable chromatin opening elements may also be used in the embodiments of the present invention.

### [IRES (Internal Ribosome Entry Site)]

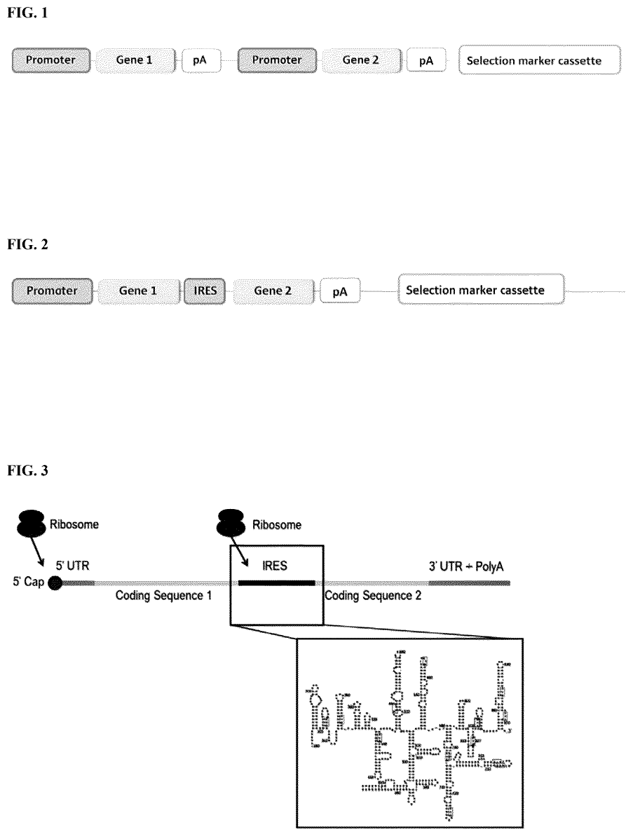

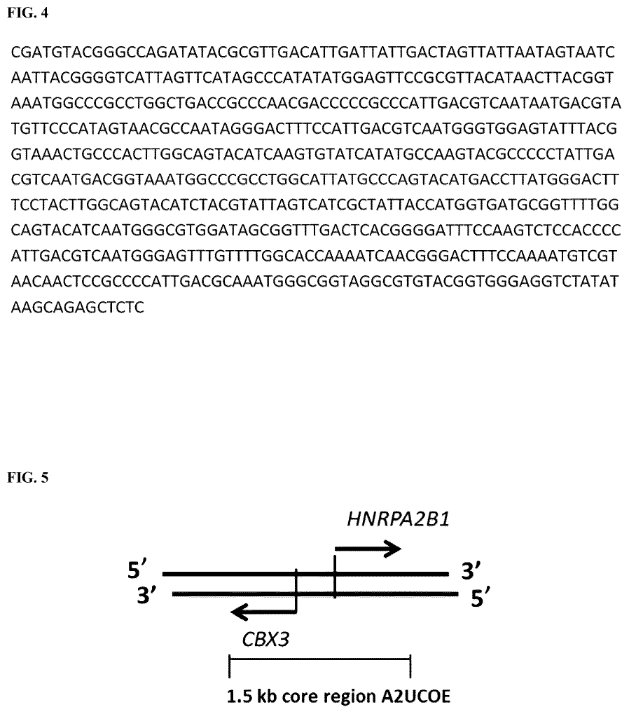

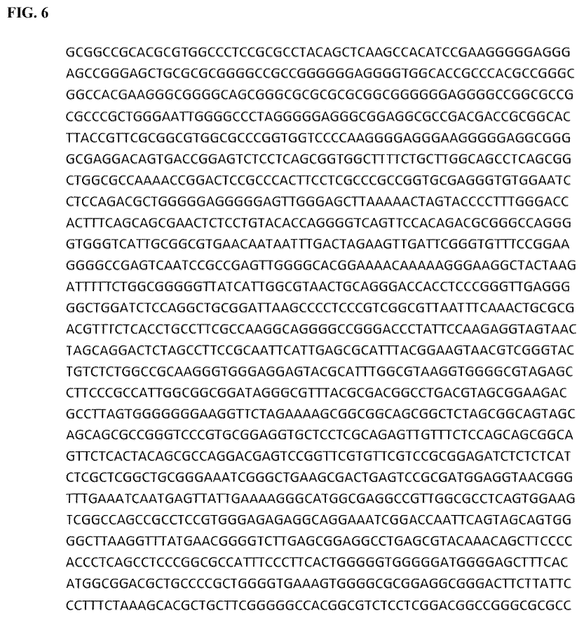

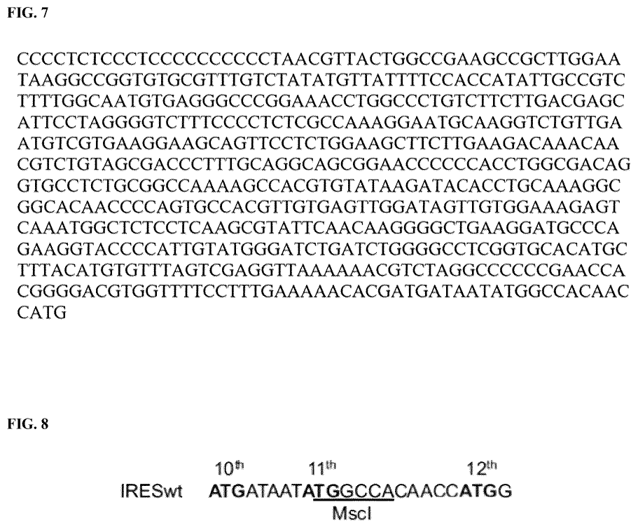

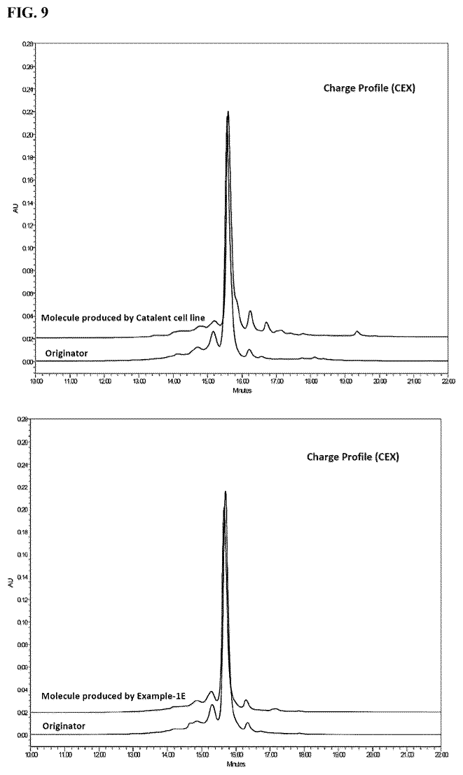

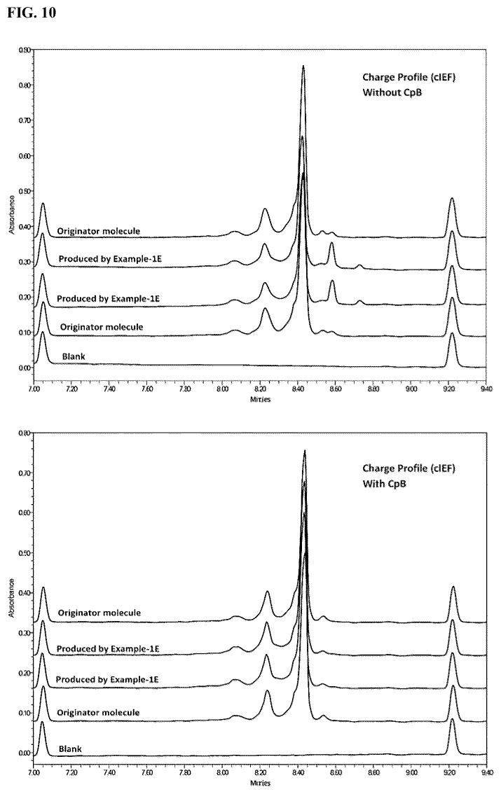

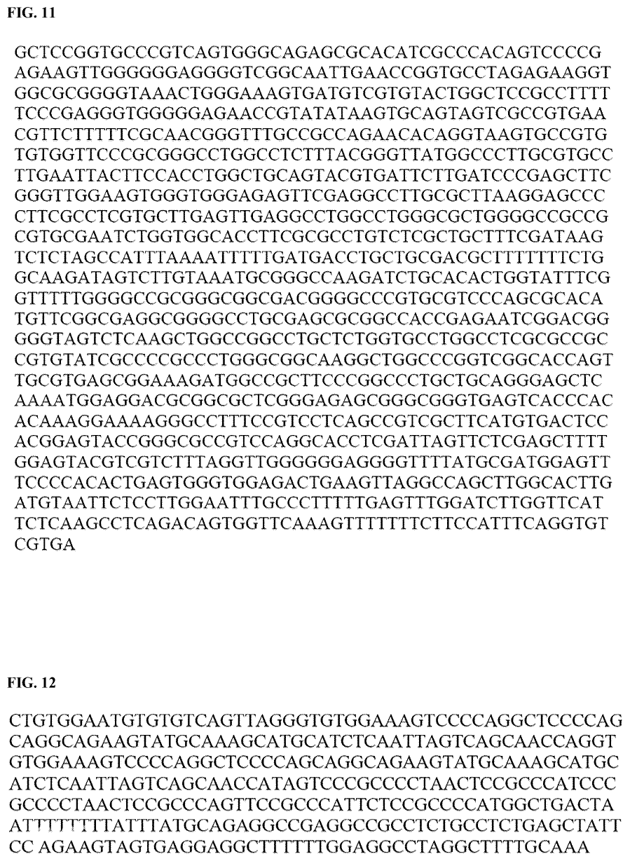

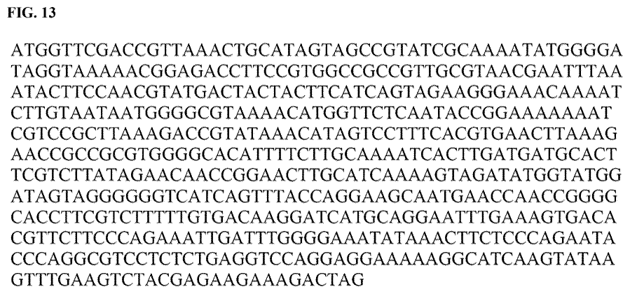

The EMCV (encephalomyocarditis virus) IRES is a non-coding RNA fragment capable of initiating high levels of cap-independent protein synthesis in mammalian cells and cell-free extracts. The advantage of using the IRES element is that two genes can be expressed from a single mRNA. The first gene is expressed by cap-dependent translation and the second gene is expressed by cap-independent binding of ribosome to the IRES sequence. The IRES sequence creates a complex secondary structure and allows the mammalian ribosome to bind and initiate translation. The secondary structure of IRES is depicted in FIG. 3.

IRES with a sequence of SEQ ID NO: 5 is one example of IRES that may be used in the present invention. Other suitable IRES sequences may also be used in the embodiments of the present invention.

### [Selection Systems]

Selection markers incorporated into plasmid vectors alongside the recombinant protein genes are usually either Glutamine synthetase (GS) or Dihydrofolate reductase (DHFR). These are two well-characterized genetic selection approaches commonly used with CHO cell lines. The plasmid vector carrying the recombinant genes and selection marker is delivered into cells by transfection, and the cells are grown under selective conditions (that is in the absence of hypoxanthine and thymine (-HT) in case of DHFR and in the absence of glutamine in case of GS). Each surviving clone will have at least one copy of the selection marker gene, with the recombinant protein gene, integrated in its genome.

### [Dihydrofolate Reductase (DHFR)]

The DHFR gene is widely used as a selectable marker in mammalian expression systems as it provides a method for amplification of the transgene. DHFR is a key enzyme in folate metabolism. De novo mitochondrial thymidylate contributes to the biosynthesis pathway and catalyzes a reaction necessary for de novo glycine and purine synthesis and DNA precursor synthesis. DHFR is a common selection marker gene used in mammalian cells and more specifically in Chinese Hamster Ovary (CHO) cells deficient in DHFR such as DG44 and DXB11.

DHFR of SEQ ID NO: 6 is one example of DHFR that may be used in the present invention. Other suitable DHFR sequences may also be used in the embodiments of the present invention.

The present invention will be described in more detail by the following Examples, but the scope of the present invention is not limited thereto. Various changes or modifications can be made by those skilled in the art based on the description of the present invention, and these changes or modifications are also included in the present invention.

### EXAMPLES

### Example 1: Vector Design

The targeted parameters during the mammalian cell line development process and the elements that were used to achieve these goals are shown in Table 1.

TABLE 1
Targeted parameters and vector elements

| | |
|---|---|
| A productive cell line | Human EF1 (eukaryotic translation elongation |
| | factor) alpha and human CMV (cytomegalovirus) |
| | promoters |
| A stable cell line | UCOE (ubiquitous chromatin opening element) |
| Less aggregation, a | EMCV (encephalomyocarditis virus) IRES |
| production process less | |
| prone to aggregation; | |
| L:H > 1 | |
| Stringent selection | Attenuated DHFR |

The designed expression vector models were obtained by integrating the obtained sequences into the template DNA. The vectors were designed using GeneART Webportal online vector design program. GeneArt Webportal has the sequence information of the elements found in their portal.

TABLE 2
Examples of designed expression vector models.

| | |
|---|---|
| Example- 1A | 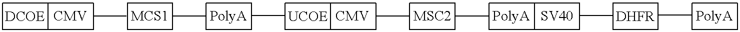 |
| Example- 1B | 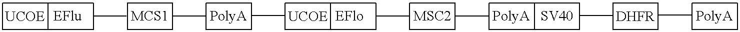 |
| Example- 1C | 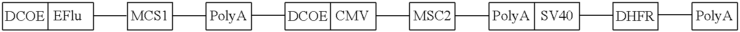 |
| Example- 1D | 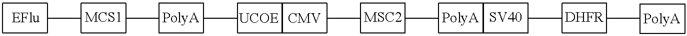 |
| Example- 1E | 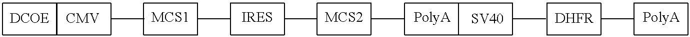 |
| Example- 1F | 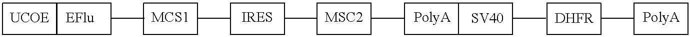 |

Example-1A: There are two CMV promoters (SEQ ID NO: 3) in the Example-1A vector mapped above. UCOE element was placed at the 5′end of both promoters. Although this model is the simplest model designed, it has the possibility of causing homologous recombination in the genome since it contains two of the same elements and promoters.

Example-1B: There are two EF1α promoters (SEQ ID NO: 2) in the Example-1B vector. There is a possibility of homologous recombination since it contains two identical elements and promoters.

Example-1C: Two different promoters, CMV and EF1α, were used in Example-1C vector.

Example-1D: Two different promoters, CMV and EF1α, were used in Example-1D vector. The CMV promoter is prone to silencing. EF 1α is more resistant to silencing due to the intron region it contains. Therefore, in Example-1D vector model, the UCOE element was only used before the CMV promoter, assuming that EF1α will remain active.

Example-1E: There is one CMV promoter (SEQ ID NO: 3) in the Example-1E vector. The IRES element was inserted between two multiple cloning sites (MCS1 and MSC2). An antibody was used as a target protein. Both the heavy chain (HC) and the light chain (LC) were expressed under the control of the CMV promoter. LC was cloned into MCS1 and HC was cloned into MCS2 to ensure that the LC:HC ratio is greater than 1.

Example-1F: There is one EF1α promoter (SEQ ID NO: 2) in the Example-1F vector. The IRES element was used and both the HC and LC were expressed under the control of the EF1α promoter. An antibody was used as a target protein. LC was cloned into MCS1 and HC was cloned into MCS2 to ensure that the LC:HC ratio is greater than 1.

### A2UCOE

The A2UCOE sequence of SEQ ID NO:4 was obtained from the NCBI epigenomics browser (accession number: NC_000007.13) [10]. A2UCOE structure is shown in FIG. 5. A2UCOE (1.5 kb) contains +309 bp from the transcription start site of CBX and +475 bp from the transcription start site of HNRPA2B1, together with the intronic region between CBX and HNRPA2B1 genes.

A2UCOE of SEQ ID NO:4 was used in the Examples provided herein.

### IRES

EMCV wild type IRES (IRESwt) of SEQ ID NO: 5 is the sequence corresponding to nucleotides 260 to 848 in the EMCV-R genome (Genbank: M81861, NC_001479; Non-Patent Literature 15). The IRESwt sequence of SEQ ID NO: 5 was used in this Example.

### [Obtaining the Designed Vector Models]

Antibody HC is inserted after IRESwt using NcoI restriction site in the 5′ region. Translation can start both from ATG-11 and ATG-12 (SEQ ID NO: 7). However, this does not affect the production of HC, because if translation is initiated from ATG-11, 4 extra amino acids forming a ‘MAAT’ site are added to the N-terminal of the signal sequence, which does not affect the cleavage of signal peptide from the correct position. This was confirmed by utilizing a Nucleofector kit V and program U-24 on a Nucleofector I system (Lonza, Cologne, Germany). Specifically, mAb heavy chain analysis was performed on the serum albumin proprotein signal sequence from the MAAT region. It was shown that it is cleaved from the right position before QV which are the first two amino acids of mAb heavy chain.

### Configuration of UCOE in Vector:

In Non-Patent Literature 14, different configurations of the UCOE element according to the position of the heavy and light chain in the vector were tested. It was shown that 5′LC 5′ HC and 3′HC 5′ LC 5′HC configurations provide the highest efficiency. Based on this information, the UCOE element was inserted at the 5′end of the promoters.

### DHFR:

DHFR of SEQ ID NO: 6 was used in the present Example.

### Example 2. Transformation, Vector Transcription and Protein Isolation

Host cells that have not successfully incorporated the vector or vector combination according to the present invention preferably die or are impaired in growth under the selective culture conditions compared to host cells that have successfully incorporated the vector or vector combination according to the present invention. During selection, host cells which have successfully incorporated the vector or vector combination can be enriched as a pool from the population of transfected host cells. Individual host cells can be isolated from the population of transfected host cells during selection and expanded, e.g., by clonal selection.

In the cell culturing step, the target protein expressed by the host cell is secreted into the culture medium. A large amount of the target protein can be obtained by purifying this secreted protein. The purification step in the present invention may include the conventional purification methods known to those skilled in the art, e.g., solubility fractionation by ammonium sulfate or PEG, ultrafiltration, fractionation by molecular weight, fractionation by various chromatography methods (for example, based on size, charge, hydrophobicity or affinity), or combinations thereof.

Once the target amino acids of the gene sequence were designed, the codons were optimized for CHO tRNA preference and submitted to Twist Bioscience for synthesis. In the present example, an antibody was used as a target protein. The weight of the heavy and light chain and the absorbance value of the twist vector were determined by gel electrophoresis. The vector was cut at the selected restriction sites (incubated with a restriction enzyme for 1 hour at 37 °C, inactivated at 80 °C for 2 hours). First, the light chain was cloned and extracted from the gel. Ligation was performed. The ligation product and the control were transferred into competent Top 10 E. coli cells by transformation. Different vector clones were collected and inoculated into separate culture vessels in 10 ml of growth medium. The cultures were incubated overnight 37 °C and agitation at 225 rpm. Mini-prep plasmid isolation was performed. After mini-prep plasmid isolation, concentrations and absorbance values of each clone were measured. Plasmids isolated from transformed colonies were digested with appropriate restriction enzymes. Inserts and digestion products were checked by agarose gel electrophoresis. PCR using specific primers was used as a secondary control. Finally, diagnostic digestion was performed with the appropriate restriction enzymes. For large-scale production, selected clones were cultured overnight in 100 mL of growth medium at 37 °C while shaking at 225 rpm. After this, the plasmid was isolated by maxi-prep and its concentration was determined.

### Example 3: Transfection and Random Integration

Vectors carrying nucleic acid sequences encoding heavy and light chains of monoclonal antibodies were transferred into E. coli cells by bacterial transformation. The transformed bacterial cells were propagated by incubation in selective agar medium. This process was performed separately for each vector. The vector expressing the monoclonal antibody was linearized by digestion with the appropriate restriction enzyme. The appropriate restriction site is located in the Ampicillin resistance gene, which is no longer required after transformation.

### Example 4: Selection of Cells Expressing the Target Antibody

### HT Selection and MTX Gene Amplification

HT selection and MTX gene amplification are performed by methods known in the art. MTX can be used to increase the gene copy number of DHFR, which often results in co-amplification of the transgene(s) for the recombinant protein of interest and can increase the overall protein productivity. MTX amplification can be performed in a single round or in multiple rounds by gradually increasing the concentration of MTX added to the selection medium.

CHO cells with DHFR deletion are transfected with recombinant DNA containing the gene of interest closely linked to the nucleotide sequence encoding DHFR. The MTX selection system is used to select CHO cells producing the protein of interest. MTX, a drug similar to folate, binds to DHFR, thereby inhibiting the production of tetrahydrofolate, which is necessary for the de-novo synthesis of purines and pyrimidines. During the gene amplification process, CHO cells are cultured in increasingly higher levels of MTX. CHO cells that have increased copies of the DHFR gene, combined with the gene of interest, are selected. CHO cells with insufficient levels of DHFR are deprived of nucleoside precursors (hypoxanthine and thymidine) and die. Once selected, transfected cell lines derived from the CHO DHFR negative host do not require MTX in the culture medium.

The DHFR-deficient strains require supplementation with glycine, hypoxanthine and thymidine. These strains were used to demonstrate that an exogenous DHFR gene could be stably transfected and selected using glycine/hypoxanthine/thymidine deficient (GHT-minus) media into cells that would otherwise be DHFR-deficient. This selection method has become a standard method for the establishment of stable transfection in CHO cell lines intended for production of therapeutic proteins. A gene that expresses the protein of interest and the DHFR gene are either combined in one mammalian expression vector or put into separate vectors. The plasmid or plasmids then are transfected into the CHO cells and the cells are grown in GHT-minus media to provide a selective environment. As a result, all surviving cells have copies of the DHFR gene and the gene of interest integrated into their genome. DHFR of SEQ ID NO: 6 was used in the present Example.

### Glutamine Synthase (GS) Selection and Methionine Sulfoxamine (MSX) Amplification

CHO cells deficient for GS, may be transfected with recombinant DNA containing the gene of interest closely linked to the gene for GS. The MSX selection system is then used to select CHO cells producing the protein of interest. MSX, a drug similar to glutamate, binds to GS, thereby inhibiting the production of glutamine, which is necessary for cell to growth. During the gene amplification process, CHO cells are cultured in high levels of MSX. CHO cells that have increased copies of the GS gene, with the gene of interest, are selected. CHO cells with insufficient levels of GS die. Once selected, transfected cell lines derived from the CHO GS negative host do not require MSX in the culture medium.

### Example 5: Limiting Dilution Cloning

CHO production cell lines are typically clonal populations derived from a single cell to ensure that the protein produced is of consistent quality. This is achieved by physically separating single cells from the heterogeneous stably transfected cell population into separate culture vessels and allowing these cells to grow into clonal cell populations. The challenge with single cell growth is that mammalian cells grow slowly or even do not survive when cultivated at low cell densities in protein-free media, which in turn leads to poor efficiencies in this process of single-cell cloning.

Procedure for single cell cloning and expansion is described herein. This procedure is used to select single clones from a pool of CHO DG44 cells which stably produce the interested protein.

Cells were seeded in the 96-well plates. Since the initial cell density is too high, firstly the cell were diluted with a dilution factor of 1:100. The amount needed for 100 wells times 30 plates was prepared. 50 μl of 1:100 cell suspensions was added to the control wells for a total volume of 200 μl. After 4 hours, imaging was performed. This is an important step for the CSI Imaging experiments because the focus is determined at this step. In order to have a good focus multiple cells are needed instead of one; for that reason, control wells in 96 well plates containing approximately 500 cells at the initial seeding were included. Once the focus was chosen, it was used for every well in the 30 96-well plates. Imaging was also performed on day 7 and 14 using the same focus. It is very important to choose a good and general focus value which will be proper with all the wells. 2-3 days after seeding, a predetermined volume (usually 60 μl) of media was collected from selected wells for the productivity test. The productivity test at this step was performed for a quick screening of the selected clones. If protein concentration was higher than 10 μg/ml, the media was diluted with the sample diluent (usually 1:1).

### Example 6: Process Development & Titer

CHO cells have become the favorite expression system for large scale production of complex biopharmaceuticals. However, industrial strategies for upstream process development are based on empirical results, due to the lack of fundamental understanding of intracellular processes.

Cell culture media and culture method play significant roles in determining optimal growth and productivity in any culture system. In order to ensure optimal performance over a wide variety of CHO cell origins, the media and feeds were derived from a library of over 70 chemically defined media and 40 chemically defined feeds based on historical performance and nutritional diversity as assessed by multivariate data analysis. Using these criteria, we identified four basal media and four new feeds that support excellent growth and productivity across a diverse set of clones.

Adaptation of cells to serum-free environment helps cells to adapt easily to suspension growth. Competent Top 10 E. Coli was prepared with CaCl₂)transformation. Two colonies were picked and seeded in 10 ml LB broth. Restriction digestion was performed with two enzymes followed by agarose gel electrophoresis. In this way, plasmid isolation was performed.

Suspension adaptation of CHO cells was achieved by using chemically defined media used in production such as PF CHO, Excell Advanced media. Adaptation of CHO cells to suspension was tested by seeding directly into serum-free medium in a shake flask (approximately 1 month). Suspension adaptation of CHO cells was checked in a sequential manner in which increasing amounts of CD medium are included in each step. CHO DG44 cells were frozen in P20 in 5%, 2.5%, 1% FBS adapted conditions.

CHO DG44 cell lines producing mAb were cultivated in 3L-15L-50L stirred tank glass bioreactors in independent fed batch processes. Daily sampling was done to measure cell density, cell viability and metabolite concentration (glucose, lactate, glutamate, glutamine, ammonium, amino acid and monoclonal antibody).

MTX gene amplification of the stable pool for production was performed after transfection. Cells were thawed, passaged and transferred into 250 ml Erlenmeyer flask to obtain enough cells for banking and several MTX amplifications. The aim of this step is to select the cells which have more copies of the expression cassette containing sequence encoding the target protein (in this case, antibody). The cells were seeded into 50, 100, 200 and 300 nM MTX containing media and passaged every 3-4 days until they became confluent and reach over 80% viability. Elisa, Glycan analysis, Resin slurry protein-A, SDS-PAGE, CEX VCD & Viability assays were performed and results analyzed. Biosimilarity analysis was performed. Batch cultures were employed to produce enough product for the analysis. One vial from two selected pools were thawed for limiting dilution cloning (LDC) and passaged once more in order to increase the viability above 95%. LDC was done by diluting cells 1:100 (1:10 and 1:10) and by seeding 0.5 cell/well. Cloning media was sterilized by filtration using the 0.22 μm coming bottle top filter. Cloning media should be filtered because the particles in FBS, which could not be seen by eye but seen under microscopy, prevents single cell imaging in 96-well plate. Different concentrations of FBC were established for LDC-Clone evaluation. Elisa, Glycan analysis, Resin slurry protein-A, SDS-PAGE, CEX VCD & Viability were performed and results analyzed.

Overall, the selected media and feeds provided excellent growth and productivity across all the clones tested. The data also supported equivalent or superior performance in selected media and feeds when compared to competitor products.

### Example 7: Genetic Stability of the Clones

Cell line and process stability are important for the manufacturing of pharmaceutical biologics. A core concept of cloning is that the genetic features of a given clone are inherited unchanged or nearly unchanged by the progeny of the cloned cells, benefiting from advantageous features identified in the clone. Initially, the emerging population remains closely related to the CHO MS of one member of a prior CHO Quasi-Species family (the clonal cell); possibly chosen for its superior specific productivity, but it may have a non-satisfying growth rate. The time frames necessary to expand this early population for Master Cell Bank generation, seed train cultures, scale-up cultures, and subsequent production phases are very substantial. During these time frames, a number of different culture conditions are applied and also different media compositions are used—before the cells arrive in a fully controlled stirred tank bioreactor with pH, oxygen, osmolality, etc.—all under scrutiny of quality by design principles. During these phases, the cells of this new CHO Quasi-Species family are evolving and could drift away from the initial CHO MS. cGMP-imposed stability studies, executed from the Master Cell Bank over several months, provide some insights into such trends, but surely will not prevent them.

Considering the process and method variability between P0 and P21, an acceptable 22% titer difference was observed. Stability of the clone cannot be assumed without quality data. This is a key factor in regulatory aspect to maintain consistent quality.

Culturing cells for 60 generations is important for genetic stability. 0th generation is called Research Cell Bank (RCB) generation, the first 12 generations are called Master Cell Bank (MCB), the next 12 generations are called Working Cell Bank (WCB) generation.

### COMPARATIVE EXAMPLE

The platforms developed within the scope of the invention obtained with several different transcriptional genetic elements and their combinations were compared with a reference innovator molecule and the molecule obtained using the Catalent cell line.

TABLE 3
Purification differences examined for a biosimilar production
process of Catalent cell line and Example-1E cell line.
Purification Differences

| Purification | Biosimilar produced by | Biosimilar produced by |
|---|---|---|
| Step | CATALENT cell line | Example-1E cell line |
| Clarification | 2 step clarification | 1 step clarification |
| Protein A | >95% Recovery - 690 | >95% Recovery - 1000- |
| Chromatography | ppm HCP | 2000 ppm HCP |
| | HCP content depends on | HCP content depends on |
| | media & feed | media & feed |
| | combination | combination |
| Polishing I | 6 different CEX resin (4 | MMIEX Chrom >90-95% |
| Chromatography | different vendor) | recovery <50 ppm HCP |
| | 3 different MMIEX (2 | |
| | different vendor) <20% | |
| | recovery when the charge | |
| | profile fit with the | |
| | originator <100 | |
| | ppm HCP | |
| Polishing II | 2 different AEX resin - 1 | not applied |
| Chromatography | MMIEX (1 vendor) | no need |
| | not catches the proper | |
| | profile. | |

Initially, transfected cells were purchased from the commercial company Catalent prior to vector design and transfection. Test studies of glycan profile, charge profile by CEX and charge profile by cIEF were carried out with biosimilar antibodies obtained from these transfected cells. However, as shown below, the biosimilar produced by Catalent cell line was quite insufficient and unsuitable for use, both in terms of glycan profile and yield. Afterwards, the CHO cells developed by the Dr. Chasin lab were provided (www.biology.columbia.edu/freeform/chasin-lab). Cloning, vector design, transfection and transformation with provided cells were performed as described above.

TABLE 4
Comparison of originator molecule, of biosimilar produced by
Catalent cell line and biosimilar produced by Example-1E.

| | | | Biosimilar | Biosimilar |
|---|---|---|---|---|
| | | | produced by | produced by |
| | Acceptance | Originator | Catalent cell | Example-1E |
| | Criteria | molecule | line | cell line |
| Glycan | G0 Group: | G0 Group: | G0 Group: | G0 Group: |
| Profile | 77.88%-98.02% | 85.3% | 82.1% | 90.7% |
| | G1 + G2 Group: | G1 + G2 Group: | G1 + G2 Group: | G1 + G2 Group: |
| | 2.13%-18.7% | 12.1% | 13.0% | 4.9% |
| | Mannose Group: | Mannose Group: | Mannose Group: | Mannose Group: |
| | 0-3.64% | 2.3% | 3.5% | 3.7% |
| | Sialic acid | Sialic acid | Sialic acid | Sialic acid |
| | Group: | Group: | Group: | Group: |
| | 0-0.2% | 0 | 1.4% | 0.12% |
| Charge | Basic Variants: | Basic Variants: | Basic Variants: | Basic Variants: |
| Profile | 4.13%-10.41% | 7.2% | 30.5% (out of limit) | 8.1% |
| by | Acidic Variants: | Acidic Variants: | Acidic Variants: | Acidic Variants: |
| CEX | 21.19%-33.63% | 26.4% | 17.5% | 26.5% |
| | Main Peak: | Main Peak: | Main Peak: | Main Peak: |
| | 58.39%-72.75% | 66.3% | 52.0% | 65.4% |
| Charge | Basic Variants: | Without | Basic Variants: | Without |
| Profile | max. 9% | Carboxypeptidase | 21.67% | Carboxypeptidase |
| by | Acidic Variants: | Basic Variants: | Acidic Variants: | Basic Variants: |
| cIEF | max. 34% | 4.1% | 14.05% | 12.9% |
| | Main Peak: | Acidic Variants: | Main Peak: | Acidic Variants: |
| | min. 59% | 20.3% | 64.28% | 22.4% |
| | | Main Peak: | | Main Peak: |
| | | 75.6% | | 64.7% |
| | | With | — | With |
| | | Carboxypeptidase | | Carboxypeptidase |
| | | Basic Variants: | | Basic Variants: |
| | | 2.2% | | 2.0% |
| | | Acidic Variants: | | Acidic Variants: |
| | | 20.6% | | 25.1% |
| | | Main Peak: | | Main Peak: |
| | | 77.3% | | 72.9% |

Two-step clarification means that 2 different filters which have large (0.8-0.4 μm) and tight (0.3-0.1 μm) nominal pore sizes were used. One-step clarification means only 1 type of filter was used. There was no difference between the production using Catalent cell line and the production using the Example-1E cell line in terms of cost and time. The tested cell lines (Catalent cell line and Example-1E) with similar capacity are different only in layout of the filters used, i.e., filter installation design.

One step clarification is easier.

In biosimilar produced by Catalent cell line, the downstream process performed as 3 chromatography mAb platform process with <20% recovery. 2 chromatography mAb platform processes could be applied with Biosimilar Produced by Example-1E Cell Line. All quality attributes are achieved. Meanwhile, 1 chromatography step is removed, the process cost and time are reduced with high recovery (>90%).

Among the biosimilarity quality criteria, glycan and charge variant profile have the greatest effect on the activity and stability of the biosimilar product.

No significant difference was observed between the two clones when evaluated in terms of glycan profile. The glycan profiles of the products gave similar results with the original product.

In the charge variant profile, which is another quality criterion, a cloning platform has been developed that enables the product to be similar to the originator product without the need for optimization. A biosimilar profile could not be obtained with the Catalent vector, and there are basic variants that not available in the originator. These basic variants that were not available in the originator could not be eliminated by both upstream and downstream process optimizations. With the designed new cloning platform, a biosimilar charge profile was obtained. According to the cIEF result, the biosimilar basic variant was higher than the originator. It was proved by Carboxypeptidase B enzyme cleavage method that the high amount of basic variant originates from C-term lysine. It is widely known in the literature that C-terminal lysines are cleaved by circulating extracellular carboxypeptidases after the product is injected into the body and thus do not pose an obstacle to product activity and biosimilarity (Non-Patent Literature 13 and Non-Patent Literature 16).

Process optimization was performed for the product developed using the Catalent vector in an attempt to improve the charge profile, which plays a critical role in the activity and stability of a biosimilar product. However, the biosimilar product charge profile could not be obtained with the originator product. In the charge variant profile obtained with the Catalent vector, process optimization studies were continued considering the risk of causing problems in the clinical stage by different variants that are not available in the originator.

On the other hand, in the product developed using the vector platform of the present invention, the charge variant profile was obtained in a biosimilar profile without the need for process optimization, and the downstream process efficiency was increased approximately 5 times, greatly reducing the cost and saving time.

### CITATION LIST

### Patent Literature

Patent literature 1: U.S. Pat. No. 4,656,134 to Ringold.
Patent literature 2: U.S. Pat. No. 5,955,422 to Lin.
Patent Literature 3: U.S. Pat. No. 5,888,774 to Delcuve.
Patent Literature 4: U.S. Pat. No. 5,605,690 to Jacobs and Smith.

### Non-Patent Literature

Non-Patent Literature 1: Coco-Martin J M., and Harmsen M M. A review of therapeutic protein expression by mammalian cells. BioProcess Int. 2008. 6:S28-S33.
Non-Patent Literature 2: Kwaks T H, Otte A P. Employing epigenetics to augment the expression of therapeutic proteins in mammalian cells. Trends Biotechnol. 2006 March; 24(3):137-42. doi: 10.1016/j.tibtech.2006.01.007. Epub 2006 Feb. 7. PMID: 16460822.
Non-Patent Literature 3: Esmer Duruel H E et al. (2021). Hucre Kulturlerine Genel Bakis. Selcuk Universitesi Fen Fakultesi Fen Dergisi. 47. 136-149.
Non-Patent Literature 4: Wurm F M. Production of recombinant protein therapeutics in cultivated mammalian cells. Nat Biotechnol. 2004 November; 22(11):1393-8. doi: 10.1038/nbt1026. PMID: 15529164.
Non-Patent Literature 5: Wurm F, Wurm M. Cloning of CHO cells, productivity and genetic stability—a discussion. Processes 2017, 5 (4): 20. doi:10.3390/pr5020020 Non-Patent Literature 6: Schimke R T et al. Amplification of dihydrofolate reductase genes in methotrexate-resistant cultured mouse cells. Cold Spring Harb Symp Quant Biol. 1978; 42 Pt 2:649-57. doi: 10.1101/sqb.1978.042.01.067. PMID: 277312.
Non-Patent Literature 7: Lee F et al. Glucocorticoids regulate expression of dihydrofolate reductase cDNA in mouse mammary tumour virus chimaeric plasmids. Nature. 1981 Nov. 19; 294(5838):228-32. doi: 10.1038/294228a0. PMID: 6272123.
Non-Patent Literature 8: Kaufman R J, Sharp P A. Amplification and expression of sequences cotransfected with a modular dihydrofolate reductase complementary dna gene. J Mol Biol. 1982 Aug. 25; 159(4):601-21. doi: 10.1016/0022-2836(82)90103-6. PMID: 6292436.
Non-Patent Literature 9: Mielke C, Maass K, Timmler M, Bode J. Anatomy of highly expressing chromosomal sites targeted by retroviral vectors. Biochemistry. 1996 Feb. 20; 35(7):2239-52. doi: 10.1021/bi952393y. PMID: 8652565.
Non-Patent Literature 10: Klehr and Bode. Mol. Genet. (Life Sci. Adv.) 1988; 7:47-52.
Non-Patent Literature 11: Chu L, Robinson D K. Industrial choices for protein production by large-scale cell culture. Curr Opin Biotechnol. 2001 April; 12(2):180-7. doi: 10.1016/s0958-1669(00)00197-x. PMID: 11287235.
Non-Patent Literature 12: Jang H P et al. In vitro and in vivo modifications of recombinant and human IgG antibodies. MAbs. 2014; 6(5):1145-54. doi: 10.4161/mabs.29883. Epub 2014 Oct. 30. PMID: 25517300; PMCID: PMC4622420.
Non-Patent Literature 14: Saunders F. et el. Chromatin function modifying elements in an industrial antibody production platform—comparison of UCOE, MAR, STAR and cHS4 elements. PLoS One. 2015 Apr. 7; 10(4):e0120096. doi: 10.1371/journal.pone.0120096. PMID: 25849659; PMCID: PMC4388700.
Non-Patent Literature 15: Koh E Y et al. An internal ribosome entry site (IRES) mutant library for tuning expression level of multiple genes in mammalian cells. PLoS One. 2013 Dec. 9; 8(12):e82100. doi: 10.1371/journal.pone.0082100. PMID: 24349195; PMCID: PMC3857217.
Non-Patent Literature 16: Cai B et al. C-terminal lysine processing of human immunoglobulin G2 heavy chain in vivo. Biotechnol Bioeng. 2011 February; 108(2):404-12. doi: 10.1002/bit.22933. PMID: 20830675.

The contents of all patent documents and non-patent documents or references explicitly cited in the present specification may be incorporated herein as part of the present specification.

1. An expression vector comprising the following elements:
a nucleotide sequence encoding one or more target proteins or peptides;
one or more terminator sequence;
one or more promoter;
a chromatin opening element (UCOE), a nucleotide sequence encoding an internal ribosome entry site (IRES) or a combination thereof;
and
a nucleotide sequence encoding a dihydrofolate reductase (DHFR) as a selection marker;
wherein the elements are operably linked.

2. The expression vector according to claim 1, wherein the expression vector is a bicistronic vector.

3. The expression vector according to claim 1, wherein the expression vector is a dual promoter vector.

4. The expression vector according to claim 1, wherein the one or more target protein or peptide is one or more monoclonal antibody.

5. The expression vector according to claim 1, wherein the one or more target protein or peptide is one or more of an antibody light chain (LC) and an antibody heavy chain (HC).

6. The expression vector according to claim 1, wherein the one or more promoter is a first promoter and a second promoter that are different from each other.

7. The expression vector according to claim 6, wherein the expression of the LC is controlled by the first promoter and the expression of the HC is controlled by the second promoter.

8. The expression vector according to claim 1, wherein the DHFR is a human DHFR and wherein the nucleotide sequence encoding the human DHFR is operably linked to a mouse-derived DHFR promoter.

9. The expression vector according to claim 6, wherein
the first promoter and the second promoter are each selected from the group consisting of a CMV promoter and a EF1-α promoter;
the UCOE comprises the sequence of SEQ ID NO: 4;
the nucleotide sequence encoding the IRES comprises the sequence of SEQ ID NO: 5; and
the DHFR comprises the sequence of SEQ ID NO: 6.

10. The expression vector according to claim 1, further comprising a first multiple cloning site (MCSI) and a second multiple cloning site (MCSII),
wherein the promoter is an EF1-α promoter;
wherein the one or more terminator sequence is a polyA signal sequence and a polyA SV40 terminator sequence; and
wherein the elements in the expression vector are arranged in the following order in 5′ to 3′ direction:
the UCOE, the promoter, MCSI, the nucleotide sequence encoding the IRES, MCSII, the polyA signal sequence, SV40 terminator sequence, the nucleotide sequence encoding the DHFR, and the polyA signal sequence.

11. The expression vector according to claim 1, further comprising a first multiple cloning site (MCSI) and a second multiple cloning site (MCSII),
wherein the promoter is a CMV promoter;
wherein the one or more terminator sequence is a polyA signal sequence and a polyA SV40 terminator sequence; and
wherein the elements in the expression vector are arranged in the following order in 5′ to 3′ direction:
the UCOE, the promoter, MCSI, the nucleotide sequence encoding the IRES, MCSII, the polyA signal sequence, the SV40 terminator sequence, the nucleotide sequence encoding the DHFR, and the polyA signal sequence.

12. The expression vector according to claim 11, wherein the SV40 terminator sequence comprises a sequence of SEQ ID NO: 1.

13. A cell or a population of cells, wherein the cell or the cells are transfected with the expression vector of claim 1.

14. The cell or the population of cells of claim 13, wherein the cell or the cells are a mammalian cell.

15. The cell or the population of cells of claim 14, wherein the mammalian cell is a Chinese Hamster Ovary (CHO) cell.

16. The cell or the population of cells of claim 13, wherein a mammalian cell culture process is (MTX) or in the absence of MTX.

17. A method of producing a target protein or peptide comprising culturing the cell or the population of cells of claim 13 under a condition for expressing the target protein or peptide in a culture medium.

18. The method of claim 17, wherein the cell or the population of cells is clones stably expressing the target protein or peptide.

19. The method of claim 17, wherein the cell or the population of cells is a mammalian cell.

20. The method of claim 17, wherein the target protein or peptide is a monoclonal antibody, antibody heavy chain, or antibody light chain, or a combination thereof.


SEQUENCE LISTING (7 sequences)

SEQ ID NO: 1 - DNA, 339 bp, synthetic construct
  source 1..339: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaa

SEQ ID NO: 2 - DNA, 1178 bp, synthetic construct
  source 1..1178: mol_type = other DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 3 - DNA, 613 bp, synthetic construct
  source 1..613: mol_type = other DNA
cgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctc

SEQ ID NO: 4 - DNA, 1579 bp, synthetic construct
  source 1..1579: mol_type = other DNA
gcggccgcacgcgtggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgccttagtgggggggaaggttctagaaaagcggcggcagcggctctagcggcagtagcagcagcgccgggtcccgtgcggaggtgctcctcgcagagttgtttctccagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggacttcttattccctttctaaagcacgctgcttcgggggccacggcgtctcctcggacggccgggcgcgcc

SEQ ID NO: 5 - DNA, 589 bp, synthetic construct
  source 1..589: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatg

SEQ ID NO: 6 - DNA, 564 bp, synthetic construct
  source 1..564: mol_type = other DNA
atggttcgaccgttaaactgcatagtagccgtatcgcaaaatatggggataggtaaaaacggagaccttccgtggccgccgttgcgtaacgaatttaaatacttccaacgtatgactactacttcatcagtagaagggaaacaaaatcttgtaataatggggcgtaaaacatggttctcaataccggaaaaaaatcgtccgcttaaagaccgtataaacatagtcctttcacgtgaacttaaagaaccgccgcgtggggcacattttcttgcaaaatcacttgatgatgcacttcgtcttatagaacaaccggaacttgcatcaaaagtagatatggtatggatagtaggggggtcatcagtttaccaggaagcaatgaaccaaccggggcaccttcgtctttttgtgacaaggatcatgcaggaatttgaaagtgacacgttcttcccagaaattgatttggggaaatataaacttctcccagaatacccaggcgtcctctctgaggtccaggaggaaaaaggcatcaagtataagtttgaagtctacgagaagaaagactag

SEQ ID NO: 7 - DNA, 24 bp, synthetic construct
  source 1..24: mol_type = other DNA
atgataatatggccacaaccatgg